(19) United States
(12) Patent Application Publication
(10) Pub. No.: US 2023/0421645 A1
(43) Pub. Date: Dec. 28, 2023

(54) SYSTEMS AND METHODS FOR GENERATING A USER PROFILE

(71) Applicant: SOFI HEALTH LTD, London (GB)

(72) Inventor: Kaveh Memari, London (GB)

(21) Appl. No.: 18/254,771

(22) PCT Filed: Jun. 24, 2021
(86) PCT No.: PCT/GB2021/051603

§ 371 Date: May 26, 2023

(30) Foreign Application Priority Data
Dec. 1, 2020 (GB) 2018947.8

(51) Int. Cl. H04L 67/306; A61B 5/16; A61B 5/00; G06F 3/04845; G06F 3/01; G06F 3/0488

(52) U.S. Cl. CPC H04L 67/306; A61B 5/165; A61B 5/167; A61B 5/742; G06F 3/04845; G06F 3/017; G06F 3/0488; G06F 3/013

(57) ABSTRACT

The invention provides a system for obtaining a user profile comprising one or more user sentiment metrics. The system includes a display unit adapted to display a representation of each of the one or more user sentiment metrics, each representation occupying a proportion of a display area of the display unit, and a user interface in communication with the display unit adapted to receive a user input. The system further comprises a processing unit, in communication with the display unit and the user interface, adapted to control the display unit to adjust a characteristic of the representation of a user sentiment metric based on the user input, the characteristic of the representation of a user sentiment metric representing the magnitude of the user sentiment metric and generate a user profile based on the adjusted characteristic of each representation of the one or more user sentiment metrics.

### FIELD OF THE INVENTION

The invention relates to the field of user profiling, and more specifically to the field of generating a user profile based on a user input.

### BACKGROUND OF THE INVENTION

There exist many different instances in which a user profile may need to be generated in order to capture and store data relating to a given user. In particular, in the field of health assessment and health monitoring, obtaining an accurate profile describing the user is important.

Typically, particularly in most medical scenarios, user profiles are generated by way of a questionnaire. For example, in the field of health assessment, a number of standardized questionnaires may be provided to the user in order to obtain the information needed to profile the user. Examples of standardized questionnaires include the GAD-7 form, which is used in assessing anxiety in a subject, and the PHQ9P form, which is used in assessing depression in a subject. In both of these examples, the user is asked to select a number indicating the severity of a given symptom.

One drawback of relying on questionnaires to generate a user profile is the lack of flexibility to an individual user, which is due to the need to standardize the questions to apply to as many users as possible. Further, a questionnaire of the nature of the GAD-7 and PHQ9P forms provides only a small number of discrete options for the user to select from, which means that the answers provided by the user may not be entirely accurate.

In addition, in order to obtain an updated user profile, existing practices simply rely on providing the same questionnaire to the user each time an update is required. Such repetition may be tedious for the user, which may lead to a lack of attention and consideration from the user when answering the questions, and may lead to the development of muscle memory, in which case answers may be provided without any thought on the user's part. Thus, the repeated provision of the same questionnaire to a user may therefore lead to an inaccurate user profile.

There is therefore a need for an improved manner of obtaining a user profile.

### SUMMARY OF THE INVENTION

The invention is defined by the claims.

According to examples in accordance with an aspect of the invention, there is provided a system for obtaining a user profile comprising one or more user sentiment metrics, the system comprising:
a display unit adapted to display a representation of each of the one or more user sentiment metrics, wherein each representation occupies a proportion of a display area of the display unit;
a user interface in communication with the display unit adapted to receive a user input; and
a processing unit, in communication with the display unit and the user interface, wherein the processing unit is adapted to:
   control the display unit to adjust a characteristic of the representation of a user sentiment metric based on the user input, and wherein the characteristic of the representation of a user sentiment metric represents the magnitude of the user sentiment metric; and
   generate a user profile based on the adjusted characteristic of each representation of the one or more user sentiment metrics.

The system provides a means for obtaining a variety of metrics of the user's current sentiments, or feelings, in order to generate a user profile.

The user sentiment metrics are measured based on the input of the user for manipulating a visual representation of said metric. In this way, the user sentiment metrics may be collected and measured in a manner that is simplistic and intuitive to the user, meaning that the user is more likely to provide a more complete and comprehensive input, as opposed to a conventional generic questionnaire.

Accordingly, the system generates a user profile with a more comprehensive and accurate dataset of user sentiment metrics.

Put another way, the system provides a means of obtaining one or more user sentiment metrics for dynamic and variable sentiment analysis based on visual cues.

In an embodiment, the characteristic of a representation comprises one or more of:
a visual characteristic comprising one or more of:
   a proportion of the display area occupied by the representation, wherein the proportion of the display area occupied by the representation of a user sentiment metric represents the magnitude of the user sentiment metric, and wherein generating the user profile is based on the adjusted proportions of the display area occupied by each representation of the one or more user sentiment metrics;
   a brightness of the representation, wherein the brightness of the representation of a user sentiment metric represents the magnitude of the user sentiment metric, and wherein generating the user profile is based on the adjusted brightness of each representation of the one or more user sentiment metrics;
   a hue of the representation, wherein the hue of the representation of a user sentiment metric represents the magnitude of the user sentiment metric, and wherein generating the user profile is based on the adjusted hue of each representation of the one or more user sentiment metrics; and
   a saturation of the representation, wherein the saturation of the representation of a user sentiment metric represents the magnitude of the user sentiment metric, and wherein generating the user profile is based on the adjusted saturation of each representation of the one or more user sentiment metrics; and/or
an audible characteristic comprising one or more of:
   a volume of an audible signal associated with the representation, wherein the volume of the audible signal associated with the representation of a user sentiment metric represents the magnitude of the user sentiment metric, and wherein generating the user profile is based on the adjusted volumes associated with each representation of the one or more user sentiment metrics; and
   a tone of an audible signal as associated with the representation, wherein the tone of the audible signal associated with the representation of a user sentiment metric represents the magnitude of the user sentiment metric, and wherein generating the user profile is based on the adjusted tones associated with each representation of the one or more user sentiment metrics.

In an embodiment, the processing unit is further adapted to:
determine a position within the display area of the display unit for each representation of the one or more user sentiment metrics; and
control the display unit to display each representation at the determined position.

In a further embodiment, for each instance of displaying the representation of each of the one or more user sentiment metrics, determining the position within the display area comprises selecting a random position within the display area for each representation. In this way, the position of the representations of the one or more user sentiment metrics may change each time they are displayed to the user, meaning that the user is not able to rely on muscle memory when providing the user input. Accordingly, user engagement and user profile accuracy may be improved.

In an embodiment, the processing unit is further adapted to:
determine a direction of approach from a starting position within the display area to the determined positon for each representation of the one or more user sentiment metrics; and
control the display unit to display each representation moving along the direction of approach to the determined position.

In a further embodiment, for each instance of displaying the representation of each of the one or more user sentiment metrics, determining the direction of approach comprises selecting a random starting position within the display area for each representation. In this way, the motion of the representations of the one or more user sentiment metrics may change each time they are displayed to the user, meaning that the user is not presented with the same view every time the system is used. Accordingly, user engagement and user profile accuracy may be improved.

In an embodiment, the user input further comprises an adjustment of a position of a representation of a user sentiment metric, and wherein the generation of the user profile is further based on the adjustment to the position of the representation.

In an embodiment, the processing unit is further adapted to perform one or more of:
add an additional user sentiment metric to the one or more user sentiment metrics based on a user input; and
remove a user sentiment metric from the one or more user sentiment metrics based on a user input. In this way, the user may tailor the system towards their individual sentiment metrics, thereby increasing the accuracy and relevance of the user profile to each individual user.

In an embodiment, a user sentiment metric of the one or more user sentiment metrics further comprises a secondary user sentiment metric, wherein the secondary user sentiment metric defines a sub-class of the user sentiment metric. In this way, each user sentiment metric may include additional detailed metrics, thereby improving the accuracy of the user profile. Secondary user sentiment metrics may be dependent on a dominant user sentiment metric and may be used to provide sub-sentiment analysis. For example, if a user sentiment metric relates to sleep, a secondary user sentiment may relate to one or more of falling asleep, staying asleep, waking up tired and the like.

In an embodiment, the user profile comprises a record of the one or more user sentiment metrics over time. In this way, changes in the one or more user sentiment metrics over time may form part of the user profile.

In an embodiment, the processing unit is further adapted to automatically adjust the characteristic of a representation of a user sentiment metric if the record of the one or more user sentiment metrics over time indicates that the user has not provided an input relating to said user sentiment metric for a predetermined period of time. In this way, the system may recognize that a user is not engaging with a given user sentiment metric, which may correlate to said user sentiment metric being of less importance to the user. Accordingly, by automatically reducing the proportion of the display area given over to the user sentiment metric that is not being engaged with, the system may aid the user in focusing on more relevant user sentiment metrics, thereby increasing the accuracy and relevance of the user profile.

In an embodiment, the processing unit is further adapted to record an order in which the user provides a user input relating to each of the one or more user sentiment metrics displayed on the display unit, thereby obtaining a user sentiment metric interaction hierarchy, and wherein the user profile further comprises the user sentiment metric interaction hierarchy. In this way, the way in which the user interacts with the system may be recorded in order to derive additional information regarding the user and the priority that the user assigns to a given user sentiment metric.

In an embodiment, the user input relating to a user sentiment metric comprises a plurality of adjustments, and wherein the processing unit is further adapted to record the plurality of adjustments, thereby obtaining an adjustment profile for the user sentiment metric, and wherein the user profile further comprises the adjustment profile. In this way, the way in which the user interacts with the system may be monitored in order to derive additional information regarding the user and the deliberation that the user has when adjusting a given user sentiment metric.

In an embodiment, a first user sentiment metric of the one or more user sentiment metrics comprises a correlation relationship with a second user sentiment metric of the one or more user sentiment metrics, and wherein the processing unit is adapted to, when adjusting the characteristic of a representation of a user sentiment metric based on the user input, adjust the characteristic of the first user sentiment metric based on the user input and adjust the characteristic of the second user sentiment metric based on the correlation relationship with the first user sentiment metric. In this way, a known relationship between user sentiment metrics may be leveraged in order to aid the user in establishing connections between one user sentiment and another when interacting with the system. For example, if a user increases a user sentiment metric relating to anxiety, a corresponding user sentiment metric relating to lack of sleep may also be increased automatically due to the known correlation between anxiety and lack of sleep. In this way, the user may be informed of links between different user sentiment metrics and the accuracy and comprehensiveness of the user profile may be increased.

In a further embodiment, the processing unit is further adapted to alter the correlation relationship based on a user input. In this way, the user may tailor the relationships between user sentiment metrics to better match their experience, which may not conform directly to a known correlation. Accordingly, the accuracy of the user profile for an individual user may be increased.

In an embodiment, the system further comprises a sensor adapted to obtain sensor data from the user, and wherein generating the user profile is further based on the sensor data, and optionally wherein the sensor comprises one or more of:
a motion senor;
a light sensor;
a sound sensor;
a heart rate sensor;
an SpO2 sensor;
a temperature sensor;
a blood sugar sensor;
a hydration sensor; and
a weight sensor.

In this way, the user input may be supplemented with additional sensor data, thereby increasing the amount of information that may be stored in the user profile and associated with the user sentiment metrics.

In a further embodiment, a first user sentiment metric of the one or more user sentiment metrics comprises a correlation relationship with a second user sentiment metric of the one or more user sentiment metrics, and wherein the processing unit is adapted to, when adjusting the proportion of the display area occupied by a representation of a user sentiment metric based on the user input, adjust the proportion of the first user sentiment metric based on the user input and adjust the proportion of the second user sentiment metric based on the correlation relationship with the first user sentiment metric, and wherein the processing unit is further adapted to alter the correlation relationship based on the sensor data. In this way, the relationships between user sentiment metrics may be adjusted automatically based on the sensor data to better match the individual user. Accordingly, the accuracy of the user profile for an individual user may be increased.

In an embodiment, the processing unit is further adapted to obtain environmental data relating to the user's environment, and wherein generating the user profile is further based on the environmental data, and optionally wherein the environmental data comprises one or more of:
geographical data;
elevation data;
weather data;
pollen count data;
humidity data;

temperature data;
pressure data;
air pollution data;
water pollution data;
light pollution data;
noise pollution data; and
UV index data.

In this way, user input may be supplemented with environmental data, thereby increasing the amount of information that may be stored in the user profile and associated with the user sentiment metrics.

In an embodiment, the user input comprises one or more of:
a hand gesture performed by the user, and optionally wherein the display unit and the user interface are incorporated into one or more of:
   a touch screen unit;
   an augmented reality unit; or
   a virtual reality unit; or
an eye movement performed by the user, wherein the system further comprises a camera adapted to capture image data of an eye of the user, and optionally wherein the display unit and the user interface are incorporated into one or more of:
   a touch screen unit;
   an augmented reality unit; or
   a virtual reality unit.

In this way, as the magnitudes of the user sentiment metrics are directly manipulated by the user in a tactile manner, the magnitude of the user sentiment metric may be more accurately and intuitively controlled.

In an embodiment, the processing unit is further adapted to generate a prompt to be provided to the user to encourage the user to provide a user input, and optionally wherein the processing unit is adapted to generate the prompt at randomized intervals. In this way, the user sentiment metrics may be captured at different times, thereby generating a more complete user profile.

In an embodiment, the user profile further comprises user demographic data. In this way, user input may be supplemented with demographic data, thereby increasing the amount of information that may be stored in the user profile and associated with the user sentiment metrics.

According to examples in accordance with an aspect of the invention, there is provided a distributed system for obtaining a plurality of user profiles comprising one or more user sentiment metrics, the distributed system comprising:
a plurality of systems as described above, each system being associated with an individual user, and wherein each of the plurality of systems further comprises a communications unit;
a remote processing unit in communication with the communication units of the plurality of systems, wherein the remote processing unit is adapted to:
   obtain a plurality of user profiles from the plurality of systems; and
   generate a community profile based on the plurality of user profiles, the community profile comprising one or more community sentiment metrics generated based on the one or more user sentiment metrics of the plurality of user profiles. In this way, a community of similar users experiencing similar user sentiment metrics may be identified.

In an embodiment, the remote processing system is further adapted to perform one or more of:
link a user profile to the community profile if the one or more user sentiment metrics of the user profile match the one or more community sentiment metrics within a predetermined tolerance;
where the user profile comprises user demographic data and the community profile further comprises community demographic data, link a user profile to the community profile if the user demographic data matches the community demographic data within a predetermined tolerance;
where the user profile comprises environmental data and the community profile further comprises community environmental data, link a user profile to the community profile if the environmental data matches the community environmental data within a predetermined tolerance; and
where the user profile comprises sensor data and the community profile further comprises community sensor data, link a user profile to the community profile if the sensor data matches the community sensor data within a predetermined tolerance; and
optionally wherein, the processing unit of each system associated with a user profile linked with a community profile is adapted to cause the display unit to display a representation of the one or more community sentiment metrics. In this way, a user profile may be automatically linked to a community profile, which may then be used to predict further information relating the user associated with said user profile.

According to examples in accordance with an aspect of the invention, there is provided a method for profiling a user based on one or more user sentiment metrics, the method comprising:
displaying a representation of each of the one or more user sentiment metrics to the user by way of a display unit, wherein each representation occupies a proportion of a display area of the display unit;
receiving a user input;
adjusting a characteristic of the representation of a user sentiment metric based on the user input, and wherein the characteristic of the representation of a user sentiment metric represents the magnitude of the user sentiment metric; and
generating a user profile based on the adjusted characteristic of each representation of the one or more user sentiment metrics.

According to examples in accordance with an aspect of the invention, there is provided a computer program comprising computer program code means which is adapted, when said computer program is run on a computer, to implement the method described above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows a schematic representation of a system for obtaining a user profile comprising one or more user sentiment metrics;

FIGS. 2A to 2D show examples of the characteristics of the representations of the one or more user sentiment metrics that may be adjusted by a user;

FIG. 3 shows an example of a system for obtaining a user profile according to an aspect of the invention;

FIG. 4 shows a schematic representation of a plurality representations of interrelated primary user sentiment metrics and secondary user sentiment metrics;

FIG. 5 shows a schematic representation of a system for obtaining a user profile according to a further aspect of the invention;

FIG. 6 shows a distributed system for obtaining a plurality of user profiles comprising one or more user sentiment metrics; and

FIG. 7 shows a method of the invention.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a system for obtaining a user profile comprising one or more user sentiment metrics. The system includes a display unit adapted to display a representation of each of the one or more user sentiment metrics, each representation occupying a proportion of a display area of the display unit, and a user interface in communication with the display unit adapted to receive a user input.

The system further comprises a processing unit, in communication with the display unit and the user interface, adapted to control the display unit to adjust a characteristic of the representation of a user sentiment metric based on the user input, the characteristic of the representation of a user sentiment metric representing the magnitude of the user sentiment metric and generate a user profile based on the adjusted characteristic of each representation of the one or more user sentiment metrics.

In other words, there is provided a system for obtaining a user profile generated based on a user input that adjusts a characteristic of a representation of a user sentiment metric, the characteristic being representative of the magnitude of the user sentiment metric represented by the representation.

Put another way, the system provides a means of profiling a user based on the user's adjustment of characteristics, or aspects, of representations of one or more user sentiment metrics.

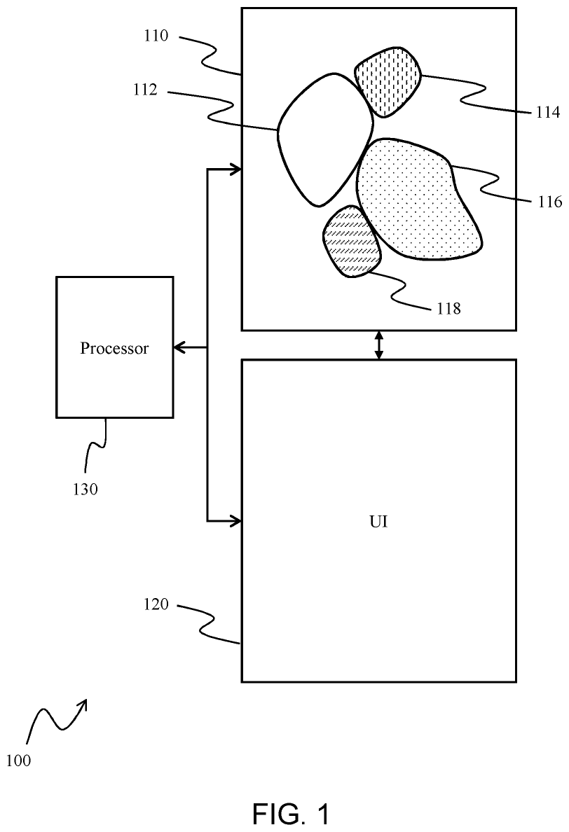

FIG. 1 shows a schematic representation of a system 100 for obtaining a user profile comprising one or more user sentiment metrics.

The system includes a display unit 110 adapted to display a representation of each of the one or more user sentiment metrics, wherein each representation occupies a proportion of a display area of the display unit. In the example shown in FIG. 1, the display unit is shown as displaying representations of four user sentiment metrics as a first representation 112, a second representation 114, a third representation 116 and a fourth representation 118. It should be noted that any number of user sentiment metrics may be displayed as individual representations via the display unit.

In the example shown in FIG. 1, the representations of the one or more user sentiment metrics are shown as bubbles. However, a representation may take any suitable form, such as one or more of: a rectangle; an ellipse; a regular polygon; an irregular polygon; a bar of a bar chart; a section of a pie chart; a cuboid; a face of a cuboid; a sphere; and the like.

The system further comprises a user interface 120 in communication with the display unit 110, the user interface being adapted to receive a user input. The user interface may be any suitable user interface that can accept a user input. For example, the user interface may comprise one or more of: a button; a switch; a scroll wheel; a scroll ball; a dial; a slider; a microphone, in which case the user input may comprise a voice command; and the like.

In a further example, the user interface and the display unit may be integrated into one or more of: a touch screen unit; an augmented reality unit; or a virtual reality unit. In these examples, the user input may comprise a hand gesture. The hand gesture may, for example, comprise one or more of: a pinching motion, wherein the user bringing their fingers together may reduce the characteristic of the representation and the user moving their fingers apart may increase the characteristic of the representation; a tapping motion, wherein tapping inside the representation may increase the characteristic and tapping outside of the representation may decrease the characteristic; a swiping motion, wherein swiping in an upward motion may increase the characteristic of the representation and swiping in a downward motion may decrease the characteristic of the representation; an elongated pressing motion, wherein an elongated press within the representation may increase the characteristic.

Further, if the system in these examples further comprises a camera, the user may provide a user input by way of an eye movement, for example by tracking the movement or focal point of the user's eye in the image data captured by the camera. In the case of the user input being provided by way of a hand gesture or an eye movement, the user adjusts the magnitudes of the user sentiment metrics by way of adjusting the characteristic of the representation in a direct and tactile manner. Accordingly, the adjustment may be made more accurately and fine tuned to a greater degree, particularly when compared to a questionnaire with set discrete answers.

The system 100 further comprises a processor 130, in communication with the display unit 110 and the user interface 120. The processing unit is adapted to control the display unit to adjust a characteristic, representing the magnitude of the user sentiment metric, of the representation of a user sentiment metric based on the user input and generate a user profile based on the adjusted characteristic of each representation of the one or more user sentiment metrics.

The system provides a means of profiling a user based on how they interact with representations of the one or more user sentiment metrics. The direct and tactile interaction with the representations of the one or more user sentiment metrics provides more granular scale on which the user may indicate the magnitude of a given user sentiment, i.e. how strongly they are feeling a certain sentiment, compared to standardized questionnaire with a discrete series of possible answers.

The user sentiment metrics are measured based on the input of the user for manipulating a visual representation of said metric, which is simplistic and intuitive to the user. Accordingly, the user is more likely to provide a more complete and comprehensive input, which may be used to generate a user profile with a more comprehensive and accurate dataset of user sentiment metrics.

The user sentiment metrics may include any sentiment, or feeling, that the user may be experiencing. For example, the one or more user sentiment metrics may include: a sleep metric; an anxiety metric; an energy metric; a stress metric; a happiness metric; a sadness metric; an anger metric; an excitement metric; a relaxation metric; a hunger metric; a thirst metric; a satisfaction metric; a pain metric; a discomfort metric; and the like.

The user sentiment metrics may be continuous metrics, in which case the user sentiment metrics may be referred to as continuous user sentiment metrics. By manipulating the visual representation of the user sentiment metrics, the user may adjust the characteristics of the user sentiment metrics on a continuous scale. In this way, the user sentiment metrics may further provide a more granular scale on which the user may more accurately indicate the magnitude of a given user sentiment, compared to conventional means of indicating user sentiment, which often rely on a discrete series of possible answers or values to represent a given metric.

In the example shown in FIG. 1, four representations of different user sentiment metrics are shown as being displayed on the display unit 110. For example, the first representation 112 may represent a sleep metric, the second representation 114 may represent an anxiety metric, the third representation 116 may represent an energy metric and a fourth representation 118 may be a stress metric.

As each user is likely to experience a diverse range of sentiments, the processing unit may be further adapted to add or remove a user sentiment metric to the one or more user sentiment metrics based on a user input. For example, a user may not be experiencing issues with their energy levels, but may be struggling to control their hunger over the course of a day. Accordingly, the user may indicate to the system, by way of a user input, that the representation of the energy metric should be removed and that a representation of a hunger metric should be added.

Put another way, the user may tailor the system towards profiling their individual sentiment metrics, thereby increasing the accuracy and relevance of the user profile to each individual user.

Further, the user profile may comprise a record of the one or more user sentiment metrics over time. In other words, the user sentiment metrics, and their change over time, may be recorded by the user interacting with the system on multiple separate occasions.

For example, a user may interact with the system to adjust the representations of the one or more user sentiment metrics, which the user may have selected as being relevant to themselves, one or more times per day. The adjustments made to the user sentiment metrics over time, such as the way in which a user's anxiety levels differ over the course of a week or longer, may form a useful dataset as part of the user profile. The more frequently a user interacts with the system, the more detailed the user profile, and in particular the way in which the user's sentiments change over time, will be. For example, an hourly record of a user's sentiment will provide a more accurate depiction of the user's true sentiments over time than a weekly or daily record. By providing the user with a simplistic and intuitive manner of recording detailed information regarding a user sentiment metric, the user is far more likely to interact with the system on a regular and frequent basis.

In addition, the processing unit may be further adapted to generate a prompt to be provided to the user to encourage the user to provide a user input. For example, the prompt may be provided to the user at regular intervals, such as one or more times a day, or at randomized intervals. By prompting the user at randomized intervals, the user sentiment metrics may be captured across a spread of different times, thereby generating a more complete user profile. Further, the prompt may be directed to a particular user sentiment metric.

The system may be incorporated into any suitable device, such as: a smartphone; a smart watch; a smart home device; a computer; a laptop; a tablet; and the like.

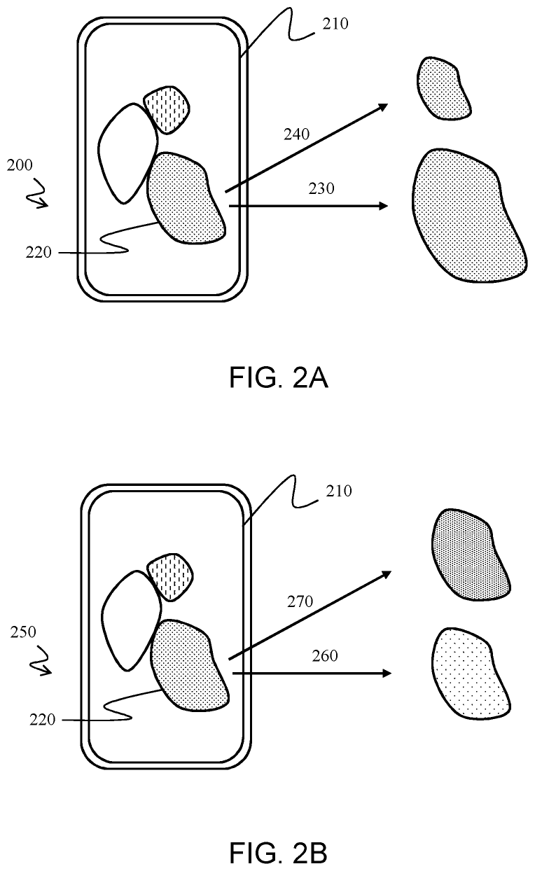

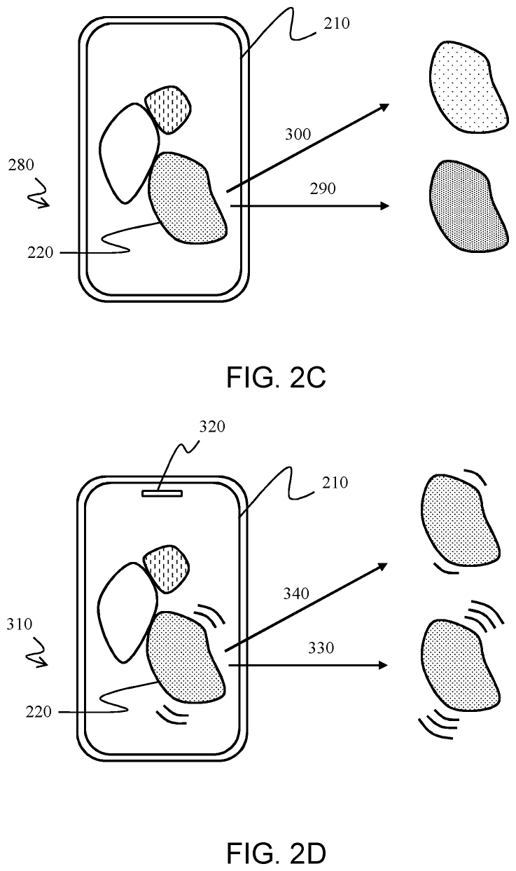

The characteristic of the representation of the one or more user sentiment metrics that may be adjusted by the user may take any suitable form. Several examples of possible adjustable characteristics of the representations are discussed below with reference to FIGS. 2A to 2D.

FIG. 2A shows an example of a system 200 for obtaining a user profile comprising one or more user sentiment metrics according to an aspect of the invention. In the example shown in FIG. 2A, the user interface and the display unit are integrated into a touch screen unit 210 and the user is interacting with one representation 220 of a user sentiment metric from among a plurality of representations shown on the touch screen unit.

In the example shown in FIG. 2A, the characteristic of the representation is a visual characteristic, and more specifically, the characteristic of the representation is the proportion of the display area occupied by the representation. The proportion of the display area occupied by the representation, i.e. the size of the representation shown on the display unit, represents the magnitude of the user sentiment metric. The user profile is then generated based on the adjusted proportions of the display area occupied by each representation of the one or more user sentiment metrics.

The user may adjust the proportion of the display area occupied by a representation 220 to increase 230 or decrease 240 the proportion of the display area occupied by the representation, thereby increasing or decreasing the magnitude of the user sentiment metric represented by the adjusted representation.

FIG. 2B shows an example of a system 250 for obtaining a user profile comprising one or more user sentiment metrics according to a further aspect of the invention. In the example shown in FIG. 2B, the user interface and the display unit are integrated into a touch screen unit 210 and the user is interacting with one representation 220 of a user sentiment metric from among a plurality of representations shown on the touch screen unit.

In the example shown in FIG. 2B, the characteristic of the representation is a visual characteristic, and more specifically, the characteristic of the representation is the brightness of the representation. The brightness of the representation of the user sentiment metric represents the magnitude of the user sentiment metric. The user profile is then generated based on the adjusted brightness of each representation of the one or more user sentiment metrics.

The user may adjust the brightness of a representation 220 to increase 260 or decrease 270 the brightness, thereby increasing or decreasing the magnitude of the user sentiment metric represented by the adjusted representation.

FIG. 2C shows an example of a system 280 for obtaining a user profile comprising one or more user sentiment metrics according to a further aspect of the invention. In the example shown in FIG. 2C, the user interface and the display unit are integrated into a touch screen unit 210 and the user is interacting with one representation 220 of a user sentiment metric from among a plurality of representations shown on the touch screen unit.

In the example shown in FIG. 2C, the characteristic of the representation is a visual characteristic, and more specifically, the characteristic of the representation is the saturation of the representation. The saturation of the representation of the user sentiment metric represents the magnitude of the user sentiment metric. The user profile is then generated based on the adjusted saturation of each representation of the one or more user sentiment metrics.

The user may adjust the saturation of a representation 220 to increase 290 or decrease 300 the saturation, thereby increasing or decreasing the magnitude of the user sentiment metric represented by the adjusted representation.

Similarly, the characteristic of the representation may be the hue of the representation, wherein the hue of the representation represents the magnitude of the user sentiment metric. The user profile may then be generated based on the adjusted hue of each representation of the one or more user sentiment metrics.

FIG. 2D shows an example of a system 310 for obtaining a user profile comprising one or more user sentiment metrics according to a further aspect of the invention. In the example shown in FIG. 2D, the user interface and the display unit are integrated into a touch screen unit 210 and the user is interacting with one representation 220 of a user sentiment metric from among a plurality of representations shown on the touch screen unit. In addition, the system further comprises a speaker unit 320 adapted to emit an audible signal in response to the user interacting the representation of a user sentiment metric.

In the example shown in FIG. 2D, the characteristic of the representation is an audible characteristic, such as the volume or tone of an audible signal associated with the representation. The audible signal may comprise a note, chord or any other sound. The audible signal may be generated when the user interacts with a representation and cease when the user stops interacting with the representation. The volume or tone of the representation of the user sentiment metric represents the magnitude of the user sentiment metric. The user profile is then generated based on the adjusted volume or tone of the audible signals associated with each representation of the one or more user sentiment metrics.

The user may adjust the volume or tone of an audible signal associated with a representation 220 to increase 330 or decrease 340 the volume or tone of the audible signal, thereby increasing or decreasing the magnitude of the user sentiment metric represented by the adjusted audible characteristic of the representation.

It should be noted that any of the characteristics described above may be used in combination with each other.

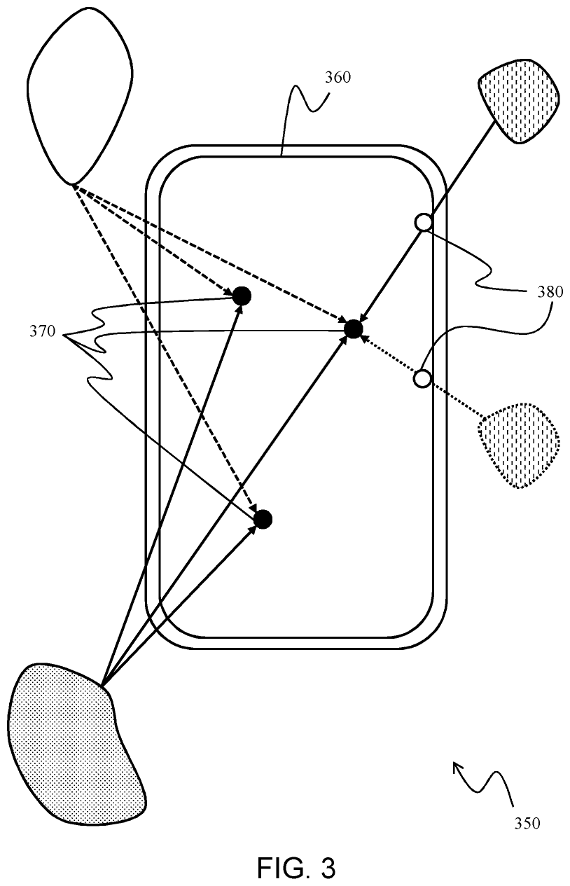

FIG. 3 shows an example of a system 350 for obtaining a user profile according to a further aspect of the invention where the user interface and the display unit are integrated into a touch screen unit 360.

In the example shown in FIG. 3, the processing unit of the system is further adapted to determine a position 370 within the display area of the display unit for each representation of the one or more user sentiment metrics. The processing unit controls the display unit to display each representation at the determined positions. The determined positions of each of the representations may be persistent across interactions with the systems. Alternatively, for each instance of displaying the representations, the position within the display area may be randomized within the display area for each representation. Put another way, the positions of the representations of the one or more user sentiment metrics may change each time they are displayed to the user, meaning that the user is not able to rely on muscle memory when providing the user input. Accordingly, user engagement may be improved and the user profile accuracy may be improved.

In addition to determining the positions within the display area for each of the representations, the processing unit may be further adapted to determine a direction of approach of the representations from a starting position 380 within the display area to the determined positon for each representation. The processor may then control the display unit to display each representation moving along the direction of approach to the determined position. As with the determined destination position 370, the starting position may be persistent across multiple interactions by the user with the system. Alternatively, the starting position may be randomized each time the user interacts with the system. Accordingly, the motion of the representations of the one or more user sentiment metrics may change each time they are displayed to the user, meaning that the user is not presented with the same view every time the system is used, which may lead to an increase in user engagement and so improve the accuracy of the user profile.

Further, the user may adjust the positions of the representations of the one or more user sentiment metrics as part of the interaction with the system. In this case, the user input may further include an adjustment of a position of a representation of a user sentiment metric. The generation of the user profile may then be further based on the adjustment of the positions of the representations by the user.

In addition to adjustments made to the characteristics of the representations of the one or more user sentiment metrics, the system may also perform automatic adjustments of the characteristics of the representations under certain criteria.

For example, the processing unit may be further adapted to automatically adjust the characteristic of a representation of a user sentiment metric if the record of the one or more user sentiment metrics over time indicates that the user has not provided an input relating to said user sentiment metric for a predetermined period of time. Put another way, if the user does not interact with a given representation of a user sentiment metric for more than a certain length of time, or number of interactions with the system, the adjustable characteristic of the representation may be adjusted such that the magnitude of the user sentiment metric is decreased. Thus, a user sentiment metric that is not strongly felt by the user will be reduced in importance, or removed, such that the user may focus on more relevant sentiments. Further, if the user sentiment metric is felt at a consistent level by the user, but the user does not regularly interact with the given representation of that metric, the automatic adjustment by the system may prompt the user to revisit this sentiment and adjust the representation accordingly, which may then also be recorded as part of the user profile.

In other words, the system may recognize that a user is not engaging with a given user sentiment metric, which may correlate to said user sentiment metric being of less importance to the user. By automatically adjusting the characteristic of the representation of the user sentiment metric that is not being engaged with, the system may aid the user in focusing on more relevant user sentiment metrics, thereby increasing the accuracy and relevance of the user profile. For example, if a user sentiment metric is not engaged with, the representation may either fade or reduce in size overtime, thereby redistributing the displayed representations to the user sentiment metrics that are actively being engaged by the user.

As discussed above, the user sentiment metrics may cover a wide variety of user sentiments and feelings, such as: a sleep metric; an anxiety metric; an energy metric; a stress metric; and the like. It is often the case that users experiencing difficulties in one of these areas will also be experiencing difficulties in another. Further, a user sentiment metric, such as a sleep metric, may have several different sub-classes of secondary metrics, such as: falling asleep or staying asleep.

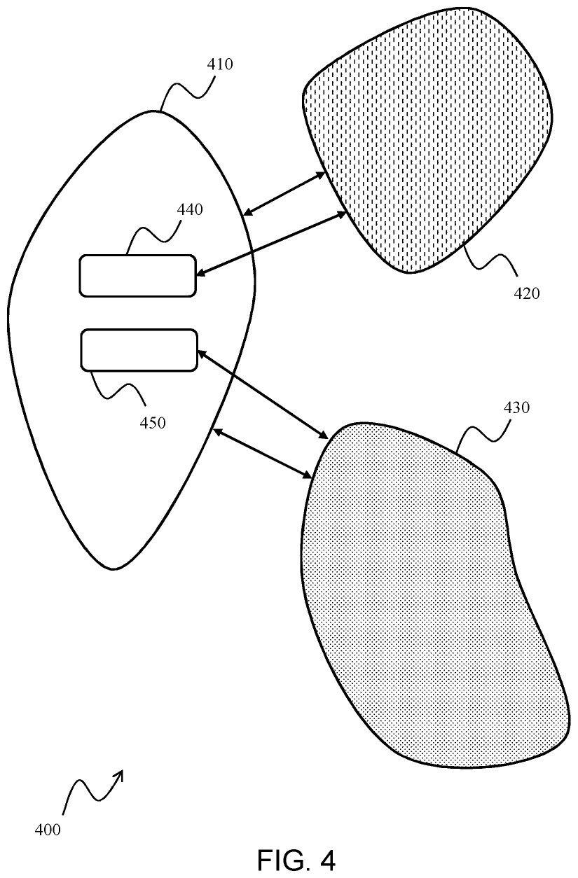

FIG. 4 shows a schematic representation 400 of a plurality representations of interrelated primary user sentiment metrics and secondary user sentiment metrics.

FIG. 4 shows three primary user sentiment metrics: a sleep metric 410; an anxiety metric 420; and an energy metric 430. In addition, FIG. 4 shows two secondary user sentiment metrics, which are sub-classes of the sleep metric 410: a going to sleep metric 440; and a staying asleep metric 450. Each user sentiment metric of the one or more user sentiment metrics that make up the user profile may comprise a plurality of secondary user sentiment metrics defining various sub-classes of the user sentiment metrics.

Put another way, each user sentiment metric may include additional, more specific metrics, thereby improving the accuracy of the user profile. Secondary user sentiment metrics may be dependent on a dominant user sentiment metric and may be used to provide sub-sentiment analysis. For example, if a user sentiment metric relates to sleep, a secondary user sentiment may relate to one or more of falling asleep, staying asleep, waking up tired and the like. The secondary user sentiment metrics may enable the user to more accurately describe their current sentiment, thereby improving the accuracy of the user profile.

As discussed above, a given user sentiment metric is not necessarily independent and may be linked with one or more other user sentiment metrics. In the example shown in FIG. 4, the sleep metric 410 is linked with both the energy metric 430 and the anxiety metric 420. This link may be established based on known clinical correlations between certain user's sentiments. For example, a user experiencing anxiety will often also be struggling with sleep, which will then cause a lack of energy in the user.

In other words, a first user sentiment metric of the one or more user sentiment metrics may comprise a correlation relationship with a second user sentiment metric of the one or more user sentiment metrics. In this case, an adjustment made to the characteristic of a representation of the first user sentiment metric based on the user input may cause a corresponding adjustment to the characteristic of the second user sentiment metric. The correlation relationship may positive, negative, linear or non-linear. The correlation relationships between the one or more user sentiment metrics may be adjusted by the user by of a user input. In this way, the user may tailor the relationships between user sentiment metrics to better match their experience, which may not conform directly to a known correlation.

Put another way, a known relationship between user sentiment metrics may be leveraged in order to aid the user in establishing connections between one user sentiment and another when interacting with the system. For example, if a user increases a user sentiment metric relating to anxiety, a corresponding user sentiment metric relating to lack of sleep may also be increased automatically due to the known correlation between anxiety and lack of sleep. In this way, the user may be informed of links between different user sentiment metrics and the accuracy and comprehensiveness of the user profile may be increased.

Further, there may also be a correlation relationship between a secondary user sentiment metric, within a primary user sentiment metric, and another user sentiment metric. In other words, the secondary user sentiment metric may have relations with other primary or secondary user sentiment metrics such that active engagement with a secondary user sentiment metric may result in an overall adjustment in the characteristics of other primary and secondary states. In the example shown in FIG. 4, the going to sleep metric 440 is linked with the anxiety metric; whereas, the staying asleep metric 450 is linked with the energy metric.

Accordingly, an adjustment made to the representation of the primary sleep metric 410 may result in an automatic adjustment of both the anxiety and sleep metrics; whereas, an adjustment to the going to sleep metric or the staying asleep metric may result in an automatic adjustment of the anxiety or sleep metric, respectively.

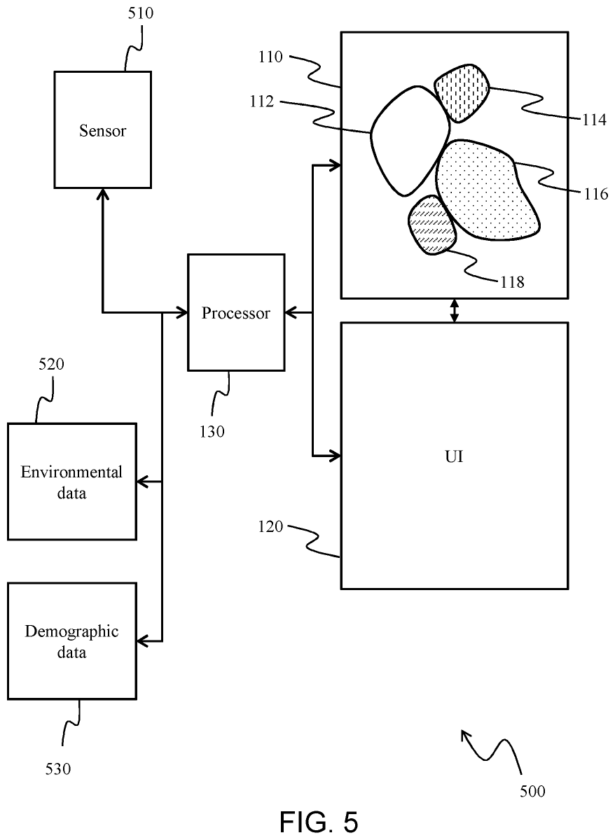

FIG. 5 shows a schematic representation of a system 500 for obtaining a user profile comprising one or more user sentiment metrics according to a further aspect of the invention.

The example system 500 shown in FIG. 5 comprises the same components as the system 100 shown in FIG. 1, which share the same reference numerals for the components described above.

Further, in the example shown in FIG. 5, the system 500 may be adapted to acquire additional data to supplement the adjusted user sentiment metrics in generating the user profile.

For example, the processing unit 130 may be further adapted to record an order in which the user provides the user input relating to each of the one or more user sentiment metrics displayed on the display unit. The recorded interaction order may be used to identify a user sentiment metric interaction hierarchy, which may form part of the user profile.

Put another way, the way in which the user interacts with the system may be recorded in order to derive additional information regarding the user and the priority that the user assigns to a given user sentiment metric. For example, in the case where the user sentiment metrics comprise an anxiety metric, an energy metric and a sleep metric, if the user interacts with the anxiety metric first, followed by the sleep metric and then the energy metric, the processing unit may place the anxiety metric at the top of the user sentiment metric interaction hierarchy followed by the sleep metric and the energy metric. In other words, the user profile may include data indicating which of the user sentiment metrics are seen as most important to the user based in the user sentiment metric interaction hierarchy.

Alternatively, or in addition, the processing unit 130 may be further adapted to record a plurality of adjustments made to a representation of a user sentiment metric, thereby obtaining an adjustment profile for the user sentiment metric. The adjustment profile may then form part of the user profile.

Put another way, the user profile may further comprise a record of the way in which the user adjusts the representation in order to arrive at the final adjusted representations of the user sentiment metrics. If the user interacts with a representation of a user sentiment metric to firstly increase the user sentiment metric, but then decreases the representation to reach the final adjustment, the user profile may include this series of adjustments in order to reflect that the user may have changed their mind when considering the given sentiment. Further, if the adjustment profile includes a large number of adjustment before the final adjustment is reached, the user profile may include a note that the user is uncertain about the given sentiment. Similarly, if the user immediately adjusts the representation of the user sentiment metric to its final adjusted form, the user profile may include a note that the user is certain about the given sentiment.

In other words, the way in which the user interacts with the system may be monitored in order to derive additional information regarding the user and the deliberation that the user has when adjusting a given user sentiment metric.

Accordingly, the processor may be adapted to derive additional data capture metrics from the user input rather than only the absolute values of the adjusted user sentiment metrics. For example, the additional data capture metrics may include one or more: the speed of adjustment of the characteristics of a representation of a user sentiment metric; the sequences of adjustments made to a representation; the sequence in which the representations are interacted with; whether a user sentiment metric is increased or decreased, all of which may provide secondary data that may form part of the user profile in addition to the final adjusted user sentiment metrics.

In the example shown in FIG. 5, the system 500 further comprises a sensor 510 adapted to obtain sensor data from the user, which may be included in the user profile in addition to the user sentiment metrics. The sensor may comprise a variety of different sensors, or sensor combinations, to obtain additional data from the user.

For example, the sensor may comprise a motion sensor adapted to obtain a motion signal from the user. The motion sensor may be any suitable motion sensor, such as an accelerometer or a gyroscope. In the case where the system in incorporated into a smartphone or smartwatch, the motion sensor may also be directly incorporated into the smartphone or smartwatch. Alternatively, the motion sensor may form part of a separate device worn by the user.

The motion signal may be used in combination with the adjusted user sentiment metrics in order to derive additional information for the user profile. For example, a user may interact to increase a user sentiment metric indicating a general issue with sleep and the motion sensor may obtain a motion signal indicating a high level of motion at night. Thus, it may be derived from the two sets of data that the user is experiencing issues with restless sleep or frequent waking, even if not explicitly recognized or noted by the user.

In a further example, the sensor may comprise a light sensor adapted to obtain a light signal. The light sensor may be any suitable light sensor, such as a camera or a photodiode. In the case where the system in incorporated into a smartphone or smart home device, the light sensor may also be directly incorporated into the smartphone or smart home device in the form of a camera. Alternatively, the light sensor may form part of a separate device in proximity of the user.

The light signal may be used in combination with the adjusted user sentiment metrics in order to derive additional information for the user profile. For example, a user may interact to increase a user sentiment metric indicating a general issue with sleep and the light sensor may obtain a light signal indicating a high level of light at night or a high level of blue light exposure prior to sleep. Thus, it may be derived from the two sets of data that the user is experiencing issues with disturbed sleep or frequent waking due to an inadequate level of darkness, even if not explicitly recognized or noted by the user.

In a further example, the sensor may comprise a sound sensor adapted to obtain a sound signal. The sound sensor may be any suitable sound sensor, such as a microphone. In the case where the system in incorporated into a smartphone, smartwatch or smart home device, the sound sensor may also be directly incorporated into the smartphone or smart home device in the form of a microphone. Alternatively, the sound sensor may form part of a separate device in proximity of the user.

The sound signal may be used in combination with the adjusted user sentiment metrics in order to derive additional information for the user profile. For example, a user may interact to increase a user sentiment metric indicating a general issue with anxiety and the sound sensor may obtain a sound signal indicating a high level of noise in the user's environment; whereas, when in a quiet environment, the user's anxiety metric is reduced. Thus, it may be derived from the two sets of data that the user is experiencing issues with anxiety due to the noise of their environment, even if not explicitly recognized or noted by the user.

In a further example, the sensor may comprise a heart rate sensor adapted to obtain a heart rate from the user. The heart sensor may be any suitable heart rate sensor for obtaining a heart rate from the user. In the case where the system in incorporated into a smartwatch, the heart rate sensor may also be directly incorporated into the smartwatch. Alternatively, the heart rate sensor may form part of a separate device in proximity of the user.

The heart rate signal may be used in combination with the adjusted user sentiment metrics in order to derive additional information for the user profile. For example, a user may interact to increase a user sentiment metric indicating a general issue with anxiety and the heart rate sensor may obtain a heart rate signal indicating an elevated heart rate. Thus, it may be derived from the two sets of data that the anxiety being experienced by the user is being understated, or overstated, by the user.

In a further example, the sensor may comprise an SpO2 sensor adapted to obtain an SpO2 signal from the user. The SpO2 sensor may be any suitable SpO2 sensor for obtaining an SpO2 reading from the user. In the case where the system in incorporated into a smartwatch, the SpO2 sensor may also be directly incorporated into the smartwatch. Alternatively, the SpO2 sensor may form part of a separate device in proximity of the user.

The SpO2 signal may be used in combination with the adjusted user sentiment metrics in order to derive additional information for the user profile. For example, a user may interact to increase a user sentiment metric indicating a general issue with energy and the SpO2 sensor may obtain an SpO2 signal indicating a low level of oxygen uptake in the user. Thus, it may be derived from the two sets of data that the user is experiencing issues with energy due to their oxygen uptake capacity, even if not explicitly recognized or noted by the user.

In a further example, the sensor may comprise a temperature sensor adapted to obtain a temperature signal. The temperature sensor may be any suitable temperature sensor, such as a thermometer. In the case where the system in incorporated into a smartphone or smartwatch, the temperature sensor may also be directly incorporated into the smartphone or smartwatch. Alternatively, the temperature sensor may form part of a separate device in proximity of the user.

The temperature signal may be used in combination with the adjusted user sentiment metrics in order to derive additional information for the user profile. For example, a user may interact to increase a user sentiment metric indicating a general issue with sleep and the temperature sensor may obtain a sound signal indicating a high temperature of the user or the user's environment. Thus, it may be derived from the two sets of data that the user is experiencing issues with sleep due to their temperature or the temperature of their environment, even if not explicitly recognized or noted by the user.

In a further example, the sensor may comprise a blood sugar sensor adapted to obtain a blood sugar signal from the user. The blood sugar sensor may be any suitable blood sugar sensor. The blood sugar sensor may be incorporated into the system or may form part of a separate device in proximity of the user.

The blood sugar signal may be used in combination with the adjusted user sentiment metrics in order to derive additional information for the user profile. For example, a user may interact to increase a user sentiment metric indicating a general issue with energy or anxiety and the blood sugar sensor may obtain a blood sugar signal indicating a low blood sugar level of the user. Thus, it may be derived from the two sets of data that the user is experiencing issues with anxiety or energy due to the user's blood sugar level, even if not explicitly recognized or noted by the user.

In a further example, the sensor may comprise a hydration sensor adapted to obtain a hydration signal from the user. The hydration sensor may be any suitable hydration sensor and may form part of the system or may form part of a separate device in proximity of the user.

The hydration signal may be used in combination with the adjusted user sentiment metrics in order to derive additional information for the user profile. For example, a user may interact to increase a user sentiment metric indicating a general issue with energy or hunger and the hydration sensor may obtain a hydration signal indicating a low hydration level. Thus, it may be derived from the two sets of data that the user is experiencing issues with energy or hunger due to the hydration of the user, even if not explicitly recognized or noted by the user.

In a further example, the sensor may comprise a weight sensor adapted to obtain a weight signal from the user. The weight sensor may be any suitable weight sensor, such as a set of scales or smart scales. In the case where the system in incorporated into a smartphone, smartwatch or smart home device, the weight sensor may be linked with a set of smart scales in order to obtain the weight signal.

The weight signal may be used in combination with the adjusted user sentiment metrics in order to derive additional information for the user profile. For example, a user may interact to increase a user sentiment metric indicating a general issue with energy and the weight sensor may obtain a weight signal indicating the user as losing, or gaining, weight. Thus, it may be derived from the two sets of data that the user is experiencing issues with energy due to the user's weight, even if not explicitly recognized or noted by the user.

In this way, the user input may be supplemented with additional sensor data, thereby increasing the amount of information that may be stored in the user profile and associated with the user sentiment metrics.

As discussed above, the user sentiment metrics may include correlation relationships between the different primary and secondary user sentiment metrics, which may be defined based on known clinical relationships. In some examples, the user sentiment metric correlation relationships may be adjusted based on the sensor data obtained by the sensors associated with the system.

In this way, the relationships between user sentiment metrics may be adjusted automatically based on the sensor data to better match the individual user.

In the example shown in FIG. 5, the system 500 is further adapted to obtain environmental data 520 relating to the user's environment, which may also be incorporated into the user profile in addition to the adjusted user sentiment metrics, and optionally the sensor data as described above.

For example, the environmental data may comprise one or more of: geographical data, such as the location of the user; elevation data; weather data; pollen count data; humidity data; temperature data; pressure data; air pollution data; water pollution data; light pollution data; noise pollution data; and UV index data.

Accordingly, the user input may be supplemented with environmental data, thereby increasing the amount of information that may be stored in the user profile and associated with the user sentiment metrics.

The environmental data may be both short-term, situational, data, such as the daily levels of each of the categories outlined above, or long-term data, such as the yearly averages of each of the categories outlined above. The long-term data may represent the long term exposure of a user to a given climate, whereas the short term data may represent the different fluctionations in the user's environment that may affect the user sentiment metrics. For example, a user living in an arid environment may experience changes to the user sentiment metrics when in a tropical environment.

In the example shown in FIG. 5, the system 500 is further adapted to obtain user demographic data 530 relating to the user's demographic information, which may also be incorporated into the user profile in addition to the adjusted user sentiment metrics, and optionally the sensor data and/or the environmental data as described above.

For example, the user demographic data may comprise one or more of: an age of a user; a gender of a user; genomic data of a user; an ethnicity of a user; a marital status of a user; an income of a user; an education of a user; an employment status of a user; internet cookie data of a user; an internet browsing history of a user; a dataset representing the musical taste of a user; a dataset representing the music listening habits of a user; a dataset representing the media consumption habits of a user; a dataset representing the social media interactions of the user; a dataset representing the purchase history of a user; a dataset representing the eating or drinking habits of a user; a dataset representing a lifestyle of a user; and the like.

In this way, user input may be supplemented with demographic data, thereby increasing the amount of information that may be stored in the user profile and associated with the user sentiment metrics.

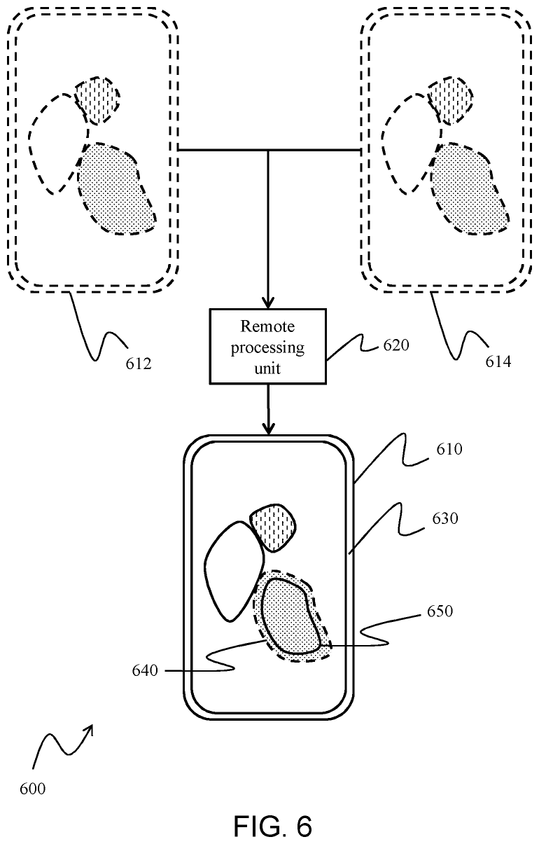

FIG. 6 shows a distributed system 600 for obtaining a plurality of user profiles comprising one or more user sentiment metrics.

The distributed system 600 includes a plurality of systems 610, 612, 614 as described above, each system being associated with an individual user. Each of the plurality of systems further comprises a communications unit.

The distributed system further includes a remote processing unit 620 in communication with the communication units of the plurality of systems. The remote processing unit is adapted to obtain a plurality of user profiles from the plurality of systems and generate a community profile based on the plurality of user profiles, the community profile comprising one or more community sentiment metrics generated based on the one or more user sentiment metrics of the plurality of user profiles.

Put another way, the distributed system may build a community of similar users experiencing similar user sentiment metrics.

The remote processing system is adapted to link a user profile to the community profile if the user profile matches the community profile within a given tolerance range. The user profile may match the community profile if the one or more user sentiment metrics of the user profile match the one or more community sentiment metrics within a predetermined tolerance. For example, if a plurality of users all share similar user sentiment metrics for anxiety, sleep and energy, they may be grouped into a community profile, wherein the community sentiment metrics may be the averaged metrics from the plurality of users. Linking the plurality of users based on the user sentiment metrics may be based on a user's current user sentiment metrics and/or the user's record of the user sentiment metrics over time. For example, if a change in a user sentiment value of an individual user matches a trend of changes within the same user sentiment metric of a community profile, the user may be linked to that community profile based on the change in the user sentiment metric.

Put another way, a change in a user sentiment metric of a user profile that matches a change in the corresponding user sentiment metric of a community profile may be used to link the user to the community experiencing the same, or a similar, change in the user sentiment metric. For example, if an individual adjusts user sentiment metric relating to anxiety to increase a given amount over a given period of time, and the anxiety metric of a given community profile increases a similar amount over a similar period of time, the user may be linked to the community profile. As the absolute values of the user sentiment metrics may vary greatly between individual users, for example due to the subjectivity of a given sentiment being measured, the accuracy of the linking of a user to a community may be improved by performing the link based on a change in a user sentiment metric rather than, or in addition to, an absolute value of the user sentiment metric.

Further, the remote processing unit may group a plurality of users into community profiles according to one or more of the data types described above, such as: user demographic data; environmental data; and sensor data. For example, users within a certain age group may be grouped together forming a community profile for users within a certain age range. In a further example, users living in a similar geographical region or climate may be grouped together forming a community profile for users experiencing similar environmental conditions. In a further example, users sharing similar sensor data may be grouped together forming a community profile based on shared sensor data. The community profile may also include a record of the number of users concurrently interacting with their associated systems.

By forming links between users based on shared data, additional, and previously unseen and unaccounted for, data linked to the user sentiment data may be derived from the user profiles. Further, users may be aggregated into a plurality of community profiles or sub-groups of community profiles for data analysis.

In the example shown in FIG. 6, the system 610 may be further adapted to cause the display unit 630 to display a representation of the one or more community sentiment metrics 640 in addition to a representation of the one or more user sentiment metrics 650.

Upon a user's first interaction with the system, the starting point of each of the user sentiment metrics may be zero or a neutral state. However, if the user provides some additional data before adjusting the user sentiment metrics, such as user demographic data, environmental data and/or sensor data, the user may first be linked to a community profile matching their provided data. In this case, the user sentiment metrics of the user may be automatically adjusted to match the average community sentiment metrics of the linked community profile for the user to then adjust according to their true sentiment.

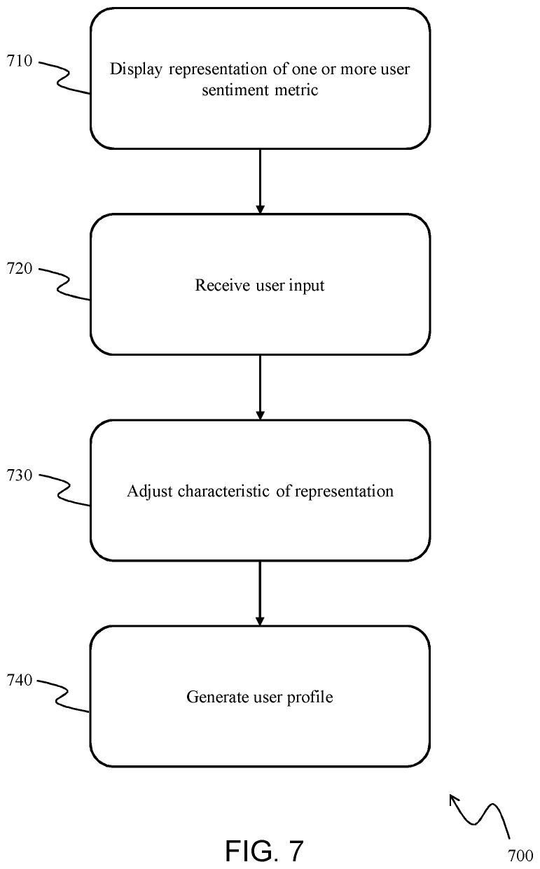

FIG. 7 shows a method 700 for profiling a user based on one or more user sentiment metrics.

The method begins in step 710 by displaying a representation of each of the one or more user sentiment metrics to the user by way of a display unit, wherein each representation occupies a proportion of a display area of the display unit.

In step 720 a user input is received and in step 730 a characteristic of the representation of a user sentiment metric is adjusted based on the user input, wherein the characteristic of the representation of a user sentiment metric represents the magnitude of the user sentiment metric.

In step 740, a user profile is generated based on the adjusted characteristic of each representation of the one or more user sentiment metrics.

The user sentiment metrics may be continuous metrics, in which case the user sentiment metrics may be referred to as continuous user sentiment metrics. By manipulating the visual representation of the user sentiment metrics, the user may adjust the characteristics of the user sentiment metrics on a continuous scale. In this way, the user sentiment metrics may further provide a more granular scale on which the user may more accurately indicate the magnitude of a given user sentiment, compared to conventional means of indicating user sentiment, which often rely on a discrete series of possible answers or values to represent a given metric.

Put another way, the user profile may be generated based on one or more continuous user sentiment metrics.

The systems and methods described above provide a significant improvement to the field of data collection and user profiling, which lead to user and community profiles comprising of far richer datasets that may be utilized in any number of applications.

For example, the systems and methods described above may be used in conjunction with a medicament dispensing device, which is adapted to dispense a medicament, such as a plant-based medicament, to a user. The medicament dispensing device may comprise a processing unit and communications unit for communicating with the systems of the invention, thereby providing additional data in the form of dispenser data, such as: dispensing frequency or amount, and medicament composition data. The user, and the community of users, may then record their user sentiment metrics in response to the dispensed medicament. The data generated by the recording of the user sentiment metrics in conjunction with data received from the medicament dispensing device may be utilized in a number of ways to increase the efficacy of the dispensed medicament for the individual users and the community of users.

A medicament may be any composition of compounds, or a compound, to be consumed by a user. For example, a medicament may be any composition of compounds adapted to alter a state or a mood of the user when consumed. A medicament may comprise one or more natural, or synthetic, compounds.

For example, analysis of the user sentiment metrics, which may be performed on an individual level by the processing unit of an individual system or a community level by the remote processing unit, may indicate that a certain medicament composition or dispensing regime is more effective to a user or group of users. In a further example, analysis of the user sentiment metrics may indicate that a certain medicament composition or dispensing regime has a greater effect on the user sentiment metrics of a user or group of users. The analysis may be performed, for example by way of a machine learning algorithm. The user, or group of users, may then be informed of this in order to enact the adjustment to the medicament composition or dispensing regime. Alternatively, such an adjustment may be automatically performed by way of a dispensing device in communication with the system of the invention.

In a further example, the systems and methods described above may be used in conjunction with a fragrance dispensing device, which is adapted to dispense a fragrance, such as a functional fragrance, to a user. The fragrance dispensing device may comprise a processing unit and communications unit for communicating with the systems of the invention, thereby providing additional data in the form of dispenser data, such as: dispensing frequency or amount, and functional fragrance composition data. The user, and the community of users, may then record their user sentiment metrics in response to the dispensed functional fragrance. The data generated by the recording of the user sentiment metrics in conjunction with data received from the fragrance dispensing device may be utilized in a number of ways to increase the efficacy of the dispensed functional fragrance for the individual users and the community of users.

A functional fragrance may be any composition of compounds, or a compound, for producing an aroma to be experienced by a user. For example, a functional fragrance may be any composition of compounds adapted to alter a state or a mood of the user when experienced, i.e. when smelled or inhaled by the user. A functional fragrance may comprise one or more natural, or synthetic, compounds.

For example, analysis of the user sentiment metrics, which may be performed on an individual level by the processing unit of an individual system or a community level by the remote processing unit, may indicate that a certain functional fragrance composition or dispensing regime is more effective to a user or group of users. In a particular example, analysis of the user sentiment metrics may indicate that a certain functional fragrance composition or dispensing regime has a greater effect on the user sentiment metrics of a user or group of users. The analysis may be performed, for example by way of a machine learning algorithm. The user, or group of users, may then be informed of this in order to enact the adjustment to the functional fragrance composition or dispensing regime. Alternatively, such an adjustment may be automatically performed by way of a dispensing device in communication with the system of the invention.

In a further example, the systems and methods described above may be used in conjunction with a dispensing device that is adapted to dispense a medicament and/or a functional fragrance to a user. The dispensing device may comprise a processing unit and communications unit for communicating with the systems of the invention, thereby providing additional data in the form of dispenser data, such as: dispensing frequency or amount, and medicament and/or functional fragrance composition data. The user, and the community of users, may then record their user sentiment metrics in response to the dispensed medicament and/or functional fragrance. The data generated by the recording of the user sentiment metrics in conjunction with data received from the dispensing device may be utilized in a number of ways to increase the efficacy of the dispensed compounds for the individual users and the community of users.

For example, analysis of the user sentiment metrics, which may be performed on an individual level by the processing unit of an individual system or a community level by the remote processing unit, may indicate that a certain medicament composition and a certain functional fragrance composition or dispensing regime is more effective to a user or group of users. In a particular example, analysis of the user sentiment metrics may indicate that a certain functional fragrance composition dispensed directly, or a predetermined length of time, after a certain medicament has been dispensed, or vice versa, has a greater effect on the user sentiment metrics of a user or group of users. The analysis may be performed, for example by way of a machine learning algorithm. The user, or group of users, may then be informed of this in order to enact the adjustment to the dispensed compositions or dispensing regime. Alternatively, such an adjustment may be automatically performed by way of a dispensing device in communication with the system of the invention.

In a further example, the systems and methods described above may be used in conjunction with a dispensing device that is adapted to dispense any composition of compounds adapted to alter a state or mood of a user. The dispensing device may comprise a processing unit and communications unit for communicating with the systems of the invention, thereby providing additional data in the form of dispenser data, such as: dispensing frequency or amount, and compound composition data. The user, and the community of users, may then record their user sentiment metrics in response to the compound. The data generated by the recording of the user sentiment metrics in conjunction with data received from the dispensing device may be utilized in a number of ways to increase the efficacy of the dispensed compounds for the individual users and the community of users.

For example, analysis of the user sentiment metrics, which may be performed on an individual level by the processing unit of an individual system or a community level by the remote processing unit, may indicate that a certain compound composition or dispensing regime is more effective to a user or group of users. The analysis may be performed, for example by way of a machine learning algorithm. The user, or group of users, may then be informed of this in order to enact the adjustment to the dispensed compositions or dispensing regime. Alternatively, such an adjustment may be automatically performed by way of a dispensing device in communication with the system of the invention.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

A single processor or other unit may fulfill the functions of several items recited in the claims.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

If the term “adapted to” is used in the claims or description, it is noted the term “adapted to” is intended to be equivalent to the term “configured to”.

Any reference signs in the claims should not be construed as limiting the scope.

1. A system for obtaining a user profile comprising a plurality of user sentiment metrics, the system comprising:
a display unit adapted to display a representation of the plurality of user sentiment metrics, wherein each representation occupies a proportion of a display area of the display unit;
a user interface in communication with the display unit adapted to receive a user input; and
a processing unit, in communication with the display unit and the user interface, wherein the processing unit is adapted to:
control the display unit to adjust a characteristic of the representation of a user sentiment metric based on the user input, and wherein the characteristic of the representation of a user sentiment metric represents the magnitude of the user sentiment metric; and
generate a user profile based on the adjusted characteristic of each representation of the plurality of user sentiment metrics.

2. A system as claimed in claim 1, wherein the characteristic of a representation comprises one or more of:
a visual characteristic comprising one or more of:
a proportion of the display area occupied by the representation, wherein the proportion of the display area occupied by the representation of a user sentiment metric represents the magnitude of the user sentiment metric, and wherein generating the user profile is based on the adjusted proportions of the display area occupied by each representation of the plurality of user sentiment metrics;
a brightness of the representation, wherein the brightness of the representation of a user sentiment metric represents the magnitude of the user sentiment metric, and wherein generating the user profile is based on the adjusted brightness of each representation of the plurality of user sentiment metrics;
a hue of the representation, wherein the hue of the representation of a user sentiment metric represents the magnitude of the user sentiment metric, and wherein generating the user profile is based on the adjusted hue of each representation of the plurality of sentiment metrics; and
a saturation of the representation, wherein the saturation of the representation of a user sentiment metric represents the magnitude of the user sentiment metric, and wherein generating the user profile is based on the adjusted saturation of each representation of the plurality of user sentiment metrics; and/or
an audible characteristic comprising one or more of:
a volume of an audible signal associated with the representation, wherein the volume of the audible signal associated with the representation of a user sentiment metric represents the magnitude of the user sentiment metric, and wherein generating the user profile is based on the adjusted volumes associated with each representation of the plurality of sentiment metrics; and
a tone of an audible signal as associated with the representation, wherein the tone of the audible signal associated with the representation of a user sentiment metric represents the magnitude of the user sentiment metric, and wherein generating the user profile is based on the adjusted tones associated with each representation of the plurality of sentiment metrics.

3. A system as claimed in claim 1, wherein the processing unit is further adapted to:
determine a position within the display area of the display unit for each representation of the plurality of user sentiment metrics; and
control the display unit to display each representation at the determined position.

4. A system as claimed in claim 3, wherein, for each instance of displaying the representation of each of the plurality of user sentiment metrics, determining the position within the display area comprises selecting a random position within the display area for each representation.

5. A system as claimed in claim 3, wherein the processing unit is further adapted to:
determine a direction of approach from a starting position within the display area to the determined position for each representation of the plurality of user sentiment metrics; and
control the display unit to display each representation moving along the direction of approach to the determined position;
and optionally wherein, for each instance of displaying the representation of each of the plurality of user sentiment metrics, determining the direction of approach comprises selecting a random starting position within the display area for each representation.

6. A system as claimed in claim 3, wherein the user input further comprises an adjustment of a position of a representation of a user sentiment metric, and wherein the generation of the user profile is further based on the adjustment to the position of the representation.

7. A system as claimed in claim 1, wherein the user sentiment metrics comprise continuous metrics.

8. A system as claimed in claim 1, wherein the processing unit is further adapted to perform one or more of:
add an additional user sentiment metric to the plurality of user sentiment metrics based on a user input; and
remove a user sentiment metric from the plurality of user sentiment metrics based on a user input.

9. A system as claimed in claim 1, wherein a user sentiment metric of the plurality of user sentiment metrics further comprises a secondary user sentiment metric, wherein the secondary user sentiment metric defines a sub-class of the user sentiment metric.

10. A system as claimed in claim 1, wherein the user profile comprises a record of the plurality of user sentiment metrics over time.

11. A system as claimed in claim 10, wherein the processing unit is further adapted to automatically adjust the characteristic of a representation of a user sentiment metric if the record of the plurality of user sentiment metrics over time indicates that the user has not provided an input relating to said user sentiment metric for a predetermined period of time.

12. A system as claimed in claim 1, wherein the processing unit is further adapted to record an order in which the user provides a user input relating to each of the plurality of user sentiment metrics displayed on the display unit, thereby obtaining a user sentiment metric interaction hierarchy, and wherein the user profile further comprises the user sentiment metric interaction hierarchy.

13. A system as claimed in claim 1, wherein the user input relating to a user sentiment metric comprises a plurality of adjustments, and wherein the processing unit is further adapted to record the plurality of adjustments, thereby obtaining an adjustment profile for the user sentiment metric, and wherein the user profile further comprises the adjustment profile.

14. A system as claimed in claim 1, wherein a first user sentiment metric of the plurality of user sentiment metrics comprises a correlation relationship with a second user sentiment metric of the plurality of user sentiment metrics, and wherein the processing unit is adapted to, when adjusting the characteristic of a representation of a user sentiment metric based on the user input, adjust the characteristic of the first user sentiment metric based on the user input and adjust the characteristic of the second user sentiment metric based on the correlation relationship with the first user sentiment metric.

15. A system as claimed in claim 14, wherein the processing unit is further adapted to alter the correlation relationship based on a user input.

16. A system as claimed in claim 1, wherein the system further comprises a sensor adapted to obtain sensor data from the user, and wherein generating the user profile is further based on the sensor data, and optionally wherein the sensor comprises one or more of:
a motion senor;
a light sensor;
a sound sensor;
a heart rate sensor;
an SpO2 sensor;
a temperature sensor;
a blood sugar sensor;
a hydration sensor; and
a weight sensor.

17. A system as claimed in claim 16, wherein a first user sentiment metric of the plurality of user sentiment metrics comprises a correlation relationship with a second user sentiment metric of the plurality of user sentiment metrics, and wherein the processing unit is adapted to, when adjusting the proportion of the display area occupied by a representation of a user sentiment metric based on the user input, adjust the proportion of the first user sentiment metric based on the user input and adjust the proportion of the second user sentiment metric based on the correlation relationship with the first user sentiment metric, and wherein the processing unit is further adapted to alter the correlation relationship based on the sensor data.

18. A system as claimed in claim 1, wherein the processing unit is further adapted to obtain environmental data relating to the user's environment, and wherein generating the user profile is further based on the environmental data, and optionally wherein the environmental data comprises one or more of:
geographical data;
elevation data;
weather data;
pollen count data;
humidity data;
temperature data;
pressure data;
air pollution data;
water pollution data;
light pollution data;
noise pollution data; and
UV index data.

19. A system as claimed in any of claims 1 to 18 claim 1, wherein the user input comprises one or more of:
a hand gesture performed by the user, and optionally wherein the display unit and the user interface are incorporated into one or more of:
a touch screen unit;
an augmented reality unit; or
a virtual reality unit; or
an eye movement performed by the user, wherein the system further comprises a camera adapted to capture image data of an eye of the user, and optionally wherein the display unit and the user interface are incorporated into one or more of:
a touch screen unit;
an augmented reality unit; or
a virtual reality unit.

20. A system as claimed in claim 1, wherein the processing unit is further adapted to generate a prompt to be provided to the user to encourage the user to provide a user input, and optionally wherein the processing unit is adapted to generate the prompt at randomized intervals.

21. A system as claimed in claim 1, wherein the user profile further comprises user demographic data.

22. A distributed system for obtaining a plurality of user profiles comprising a plurality of user sentiment metrics, the distributed system comprising:
a plurality of systems as claimed in claim 1, each system being associated with an individual user, and wherein each of the plurality of systems further comprises a communications unit;
a remote processing unit in communication with the communication units of the plurality of systems, wherein the remote processing unit is adapted to:
obtain a plurality of user profiles from the plurality of systems; and
generate a community profile based on the plurality of user profiles, the community profile comprising a plurality of community sentiment metrics generated based on the plurality of user sentiment metrics of the plurality of user profiles.

23. A distributed system as claimed in claim 22, wherein the remote processing system is further adapted to perform one or more of:
link a user profile to the community profile if the plurality of user sentiment metrics of the user profile match the plurality of community sentiment metrics within a predetermined tolerance;
where the user profile comprises user demographic data and the community profile further comprises community demographic data, link a user profile to the community profile if the user demographic data matches the community demographic data within a predetermined tolerance;
where the user profile comprises environmental data and the community profile further comprises community environmental data, link a user profile to the community profile if the environmental data matches the community environmental data within a predetermined tolerance; and
where the user profile comprises sensor data and the community profile further comprises community sensor data, link a user profile to the community profile if the sensor data matches the community sensor data within a predetermined tolerance; and
optionally wherein, the processing unit of each system associated with a user profile linked with a community profile is adapted to cause the display unit to display a representation of the plurality of community sentiment metrics.

24. A method for profiling a user based on a plurality of user sentiment metrics, the method comprising:
displaying a representation of each of the plurality of user sentiment metrics to the user by way of a display unit, wherein each representation occupies a proportion of a display area of the display unit;
receiving a user input;
adjusting a characteristic of the representation of a user sentiment metric based on the user input, and wherein the characteristic of the representation of a user sentiment metric represents the magnitude of the user sentiment metric; and
generating a user profile based on the adjusted characteristic of each representation of the plurality of user sentiment metrics.

25. A computer program comprising computer program code means which is adapted, when said computer program is run on a computer, to implement the method of claim 24.
